(19) United States
(12) Patent Application Publication
(10) Pub. No.: US 2022/0050071 A1
(43) Pub. Date: Feb. 17, 2022

(54) MULTI-PHASE WATER OIL COMPOSITION AND SALINITY METERING SYSTEM AND METHOD

(71) Applicant: BL Technologies, Inc., Minnetonka, MN (US)

(72) Inventors: William PLATT, Minnetonka, MN (US); Matthew SCHULMERICH, Minnetonka, MN (US); Cheryl SURMAN, Minnetonka, MN (US); Steven GO, Minnetonka, MN (US)

(21) Appl. No.: 17/312,659

(22) PCT Filed: Dec. 21, 2018
(86) PCT No.: PCT/US2018/067059

§ 371 Date: Jun. 10, 2021

(51) Int. Cl. G01N 27/02; G01N 33/28

(52) U.S. Cl. CPC G01N 27/026; G01N 33/2835; G01N 33/2823

(57) ABSTRACT

A vessel sensor for measuring real-time data of a multiphase fluid, the vessel sensor having a housing; an inner electrode, wherein the inner electrode is positioned within the housing; and a vessel cavity located between the housing and the inner electrode.

### FIELD OF INVENTION

The disclosed technology generally described hereinafter provides for a sensor and sensor system for measuring real-time data of a multiphase fluid, and more specifically, to a sensor and sensor system for determining salinity concentrations in multiphase fluids using impedance spectroscopy.

### BACKGROUND OF THE INVENTION

All crude oil when produced contains some amounts of emulsified salt. Treatments in the field help remove most of the salt, but significant amounts of salt in the order of 100 to 1000 pounds of salt per thousand barrels of oil are still present. Salt concentration in crude can cause fouling and corrosion of various equipment in the refinery process. For these reasons, it is important that salt content of crude oil be reduced to the lowest practical limit prior to refining.

### SUMMARY OF THE INVENTION

The disclosed technology generally described hereinafter provides for a sensor and sensor system for measuring real-time data of a multiphase fluid, and more specifically, to a vessel sensor and sensor system for determining salinity concentrations in multiphase fluids using impedance spectroscopy.

In one aspect of the present technology, a vessel sensor for measuring real-time data of a multiphase fluid is provided. The vessel sensor comprises a housing; an inner electrode, wherein the inner electrode is positioned within the housing; and a vessel cavity located between the housing and the inner electrode.

In some embodiments, the inner electrode is a tube-shaped electrode. In some embodiments, the housing is generally cylindrical. In some embodiments, the vessel sensor detects a salinity concentration of at least 0.16 lbs/1000 bbl within a multiphase fluid. In some embodiments, a multiphase fluid flows through the vessel cavity. In some embodiments, the vessel sensor provides a response to a multiphase fluid concentration. In some embodiments, the multiphase fluid is a down-hole or in-line multiphase fluid in proximity to the vessel sensor. In some embodiments, the vessel sensor measures values of real and imaginary parts of an impedance spectra associated with a multiphase fluid concentration while proximate to a multiphase fluid, and said measured values are used to determine salinity concentrations of the multiphase fluid.

In yet another aspect of the present technology, a sensor system for measuring real-time data of a multiphase fluid is provided. The sensor system comprises a vessel sensor, the vessel sensor comprising an inner electrode, an outer cylindrical body, and a cavity located between the inner electrode and the outer cylindrical body; an inductor; and an impedance analyzer.

In some embodiments, the multiphase fluid flows through the cavity located between the inner electrode and the outer cylindrical body. In some embodiments, the vessel sensor provides a response to the multiphase fluid concentration, wherein the multiphase fluid is a down-hole or in-line multiphase fluid in proximity to the vessel sensor.

In some embodiments, the vessel sensor is configured to measure values of real and imaginary parts of an impedance spectra associated with a multiphase fluid concentration while proximate to a multiphase fluid. In some embodiments, the measured values of real and imaginary parts of the impedance spectra are used to determine salinity concentrations of the multiphase fluid. In some embodiments, the multiphase fluid is oil, crude oil, desalted oil, or live oil. In some embodiments, the inductor is a high-Q resonating inductor. In some embodiments, the sensor system further comprises a processor coupled to the impedance analyzer. In some embodiments, the vessel sensor detects a salinity concentration of at least 0.16 lbs/1000 bbl within a multiphase fluid.

In yet another aspect of the present technology, a method for detecting real-time data of a multiphase fluid is provided. The method comprises providing a vessel sensor; deploying the vessel sensor within a multiphase fluid environment; measuring values of both real and imaginary parts of a complex impedance spectra associated with a multiphase fluid; and determining real-time data of the multiphase fluid. In some embodiments, the multiphase fluid is a down-hole or in-line multiphase fluid in proximity to the vessel sensor. In some embodiments, the vessel sensor comprises an outer cylindrical body, an inner electrode having an electrode, and a cavity located therebetween. In some embodiments, the measured values of real and imaginary parts of the impedance spectra associated with the multiphase fluid concentration while proximate to the multiphase fluid are used to determine salinity concentrations of the multiphase fluid. In some embodiments, the multiphase fluid environment comprises the effluent or influent of a desalter. In some embodiments, determining the real-time data of the multiphase fluid includes detecting a salinity concentration of at least 0.16 lbs/1000 bbl within the multiphase fluid.

### BRIEF DESCRIPTION OF THE DRAWINGS

These and other features of the disclosed technology, and the advantages, are illustrated specifically in embodiments now to be described, by way of example, with reference to the accompanying diagrammatic drawings, in which:

FIG. 1 is a general schematic of an illustrative embodiment of the disclosed technology;

FIG. 2 is a circuit diagram of an illustrative embodiment of the disclosed technology;

FIG. 3 is an illustrative embodiment of the disclosed technology;

FIG. 4 is an illustrative embodiment of the disclosed technology; and

FIG. 5 is a graphical representation of an illustrative embodiment of the disclosed technology.

### DETAILED DESCRIPTION OF EXEMPLARY EMBODIMENTS

The disclosed technology generally provides a sensor and sensor system for determining salt concentrations in multiphase fluids using impedance spectroscopy. The disclosed technology provides a sensor and sensor system which monitors salt levels in a refinery operation in an automatic fashion. The sensor and sensor system enables real-time monitoring of the efficiency of a desalter and provides automated feedback, thus eliminating the need for manual inspections that require highly trained and experienced technicians.

The disclosed technology provides a vessel sensor 100. The vessel sensor 100 provides instrumentation to monitor the salt levels in refinery operations automatically, thus enabling real-time monitoring of efficiency of a desalter, and providing automated feedback for emulsion breaker chemistry controls. The length of the vessel sensor is between about 100-500 mm and the radius is about 20-50 mm, where the ideal dimensions are dependent on the limit of detection and dynamic range of the salt content in the fluid under measurement.

The vessel sensor 100 is installed inline within the effluent or influent of a desalter unit and provides real-time data on the amount of salt, as well as water content, within the multiphase fluid. In some embodiments, the multiphase fluids as described herein may include, but are not limited to, oil, crude oil, desalted oil, live oil, or combinations thereof. In some embodiments, the multiphase fluid is a down-hole or in-line multiphase fluid in proximity to the vessel sensor.

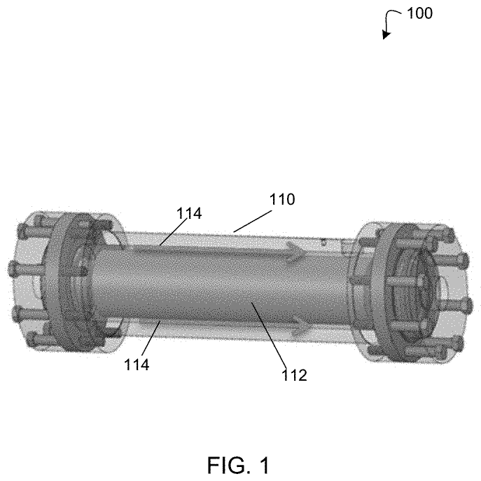

As shown in FIG. 1, the vessel sensor 100 comprises a housing 110, and an inner electrode 112, wherein the inner electrode 112 is positioned within and/or encompassed by the housing 110. The housing 110 comprises an inner housing and an outer housing, (not shown in figures). The inner electrode 112 housing is seated in a dielectric material, such that the inner housing and outer housing are not electrically connected. In some embodiments, the inner electrode 112 is a tube-shaped electrode. It should be understood by a person skilled in the art that the inner electrode 112 can be of any shape capable of being contained within the housing and being able to adequately measure the complex impedance. In some embodiments, the housing 110 is generally cylindrical. It should be understood by a person skilled in the art that the housing 110 can be of any shape or form desirable in order to be utilized within various refinery operations.

In some embodiments, the inner electrode 112 is configured to measure the complex impedance of the vessel sensor 100. The complex impedance of the vessel will change as a function of the multiphase fluid concentration. The vessel sensor 100 measures values of real and imaginary parts of an impedance spectra associated with the multiphase fluid concentration, where these measured values are then used to determine salinity concentrations of the multiphase fluid. The real and imaginary or “complex” impedance spectra are used to build a multivariable calibration model that allows for prediction of salinity. In some embodiments, the vessel sensor detects a salinity concentration of at least 0.16 lbs of salt per 1000 barrels of oil within a multiphase fluid.

A complex impedance signal is sent to the outer housing that excites the inner housing via mutual inductance. Any conductive material is suitable for the housings, but it needs to be compatable with the chemical and physical enviroment into which it is deployed.

The vessel sensor 100 further comprises a vessel cavity or annulus 114 located between the housing 110 and the inner electrode 112. The vessel cavity 114 is depicted by arrows in FIG. 1. The vessel cavity 114 allows for multiphase fluids to flow through the vessel cavity 114 where the complex impedence is measured.

During operation, the electrical capacitance varies as a function of the dielectric change within the fluid composition. Specifically, as the multiphase fluid compositions change, the dielectric properties of the fluid also change proportionally depending on the types of fluid mixtures. The capacitance (C) is proportional to the area of the inner electrode and the housing and the dielectric material between them. The capacitance is also inversely proportional to the distance between the inner electrode and the housing. This relationship can be defined by C=(A*ϵr/)d, where A is the area of the inner electrode and the housing, ϵr is the dielectric constant, and d the distance between the inner electrode and the housing. As the multiphase fluids flow between the inner electrode 112 and the housing 110, depending on the composition of the fluid, the capacitance and complex impedance will be used to model the salinity of the fluid.

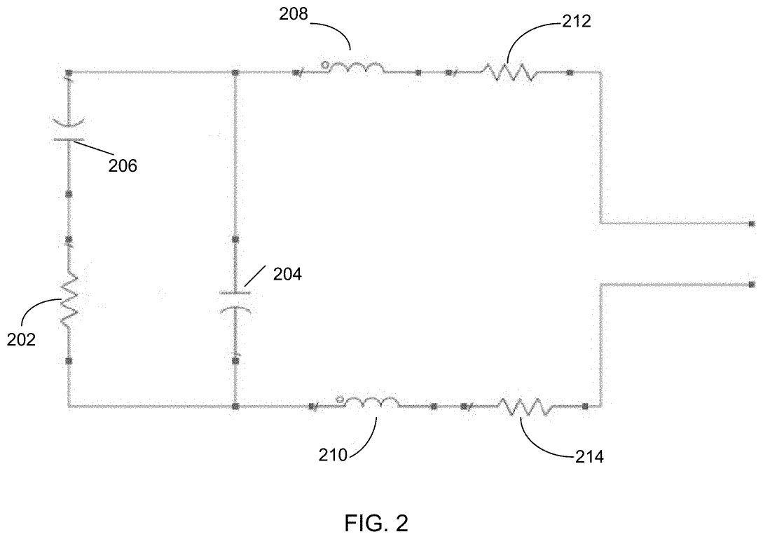

Now turning to FIG. 2, an equivalent circuit 200 of the vessel sensor 100 is provided. The equivalent circuit 200 forms an inductor-capacitor-resistor (LCR) circuit and comprises an R2 resistor 202 and C2 capacitor 204. The equivalent circuit 200 also includes C3 (parasitic) capacitor 206, L2 (parasitic) inductor 208, L3 (parasitic) inductor 210, R3 (parasitic) resistor 212, and R4 (parasitic) resistor 214.

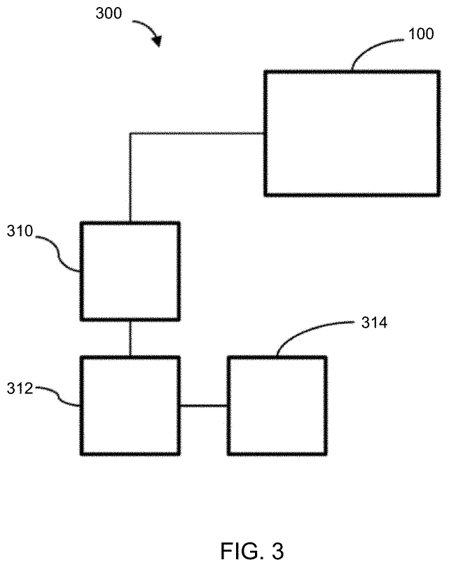

Illustrated in FIG. 3 is a schematic of an embodiment of the sensor system 300. The sensor system 300 comprises the vessel sensor 100, as previously described, as well as an inductor 310, and an impedance analyzer 312.

The inductor 310 that is electrically connected to the outer housing of the sensor vessel 100, electrically resonates the vessel sensor 100, where the complex impedance is measured by an impedance analyzer 312 to record the resonance frequency and complex impedance. In some embodiments, the inductor is a high-Q resonating inductor.

By means of resonating the vessel sensor 100 structure, the signal is increased by roughly multiplying quality factor (Q factor) of the circuit. Using the complex impedance data obtained, the salt or salinity concentration of a multiphase fluid, as for example oil, is derived from the real and imaginary part of impedance, as well as resonance frequency of the signal spectrum using multivariate modeling techniques.

In some embodiments, the impedance analyzer 312 is coupled to a processor 314 such as a microcomputer. Data received from the impedance analyzer 312 is processed using multivariable analysis, and the output may be provided through a user interface.

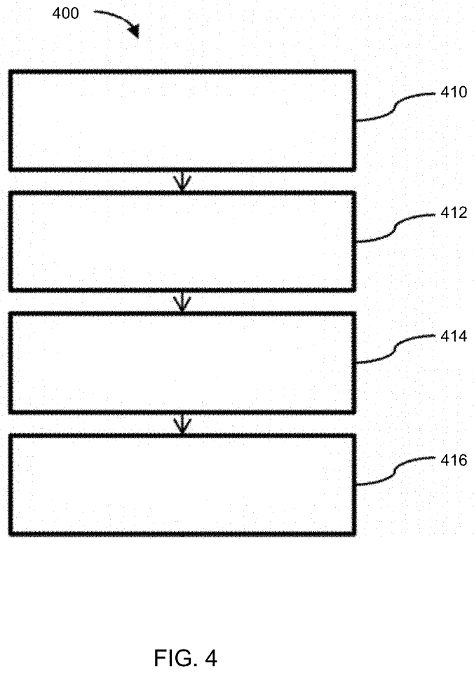

The disclosed technology further provides a method for detecting real-time data of a multiphase fluid. As shown in FIG. 4, the method 400 comprises providing a vessel sensor (step 410); deploying the vessel sensor within a multiphase fluid environment (step 412); measuring values of both real and imaginary parts of a complex impedance spectra associated with a multiphase fluid (step 414); and determining real-time data of the multiphase fluid (step 416).

In step 410, the method utilizes the vessel sensor 100 as previously described. In some embodiments, the vessel sensor 100 comprises an outer cylindrical body, an inner electrode having an electrode, and a cavity located therebetween.

In step 412, the vessel sensor 100 is deployed within a multiphase fluid environment. In some embodiments, the multiphase fluid environment, includes, but is not limited to, oil, crude oil, desalted oil, live oil, or combinations thereof. In some embodiments, the multiphase fluid environment comprises a down-hole or in-line multiphase fluid in proximity to the vessel sensor. In other embodiments, the multiphase fluid environment comprises the effluent or influent of a desalter.

In step 414, the real and imaginary parts of a complex impedance spectra associated with a multiphase fluid are measured. The measured values of both real and imaginary parts of the impedance spectra associated with the multiphase fluid concentration are used to determine salinity concentrations of the multiphase fluid.

In step 416, the real-time data of the multiphase fluid is determined. This determination is includes detecting a salinity concentration of at least about 0.16 lbs/1000 bbl within the multiphase fluid.

### EXAMPLES

The present invention will be further described in the following examples, which should be viewed as being illustrative and should not be construed to narrow the scope of the invention or limit the scope to any particular invention embodiments.

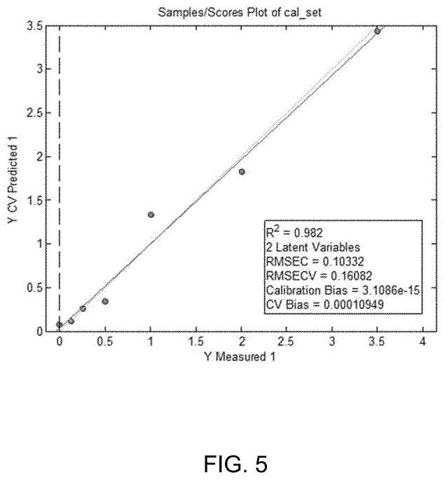

The real and imaginary spectra from the sensor were used to calibrate a multivariate model that is representative of the fluids. That training set was then used to predict samples not in the training set, and the model predictions were compared to the actual values. The results of the model performance are shown in FIG. 5.

FIG. 5 is a correlation plot that plots the measured amount salt on the X-axis vs. the predicted amount of salt on the Y-axis. (Standard Error of Validation (SEV)=0.16 ptb (lbs/1000 bbl).)

While embodiments of the disclosed technology have been described, it should be understood that the present disclosure is not so limited and modifications may be made without departing from the disclosed technology. The scope of the disclosed technology is defined by the appended claims, and all devices, processes, and methods that come within the meaning of the claims, either literally or by equivalence, are intended to be embraced therein.

1. A vessel sensor for measuring real-time data of a multiphase fluid, the vessel sensor comprising:
a housing, wherein the housing is generally cylindrical;
an inner electrode, wherein the inner electrode is positioned within the housing; and
a vessel cavity located between the housing and the inner electrode.

2. The vessel sensor as recited in claim 1, wherein the inner electrode is a tube-shaped electrode.

3. (canceled)

4. The vessel sensor as recited in claim 1, wherein the vessel sensor detects a salinity concentration of at least 0.16 lbs/1000 bbl within a multiphase fluid.

5. The vessel sensor as recited in claim 1, wherein a multiphase fluid flows through the vessel cavity.

6. The vessel sensor as recited in claim 1, wherein the vessel sensor provides a response to a multiphase fluid concentration.

7. The vessel sensor as recited in claim 5, wherein the multiphase fluid is a down-hole or in-line multiphase fluid in proximity to the vessel sensor.

8. The vessel sensor as recited in claim 1, wherein the vessel sensor measures values of real and imaginary parts of an impedance spectra associated with a multiphase fluid concentration while proximate to a multiphase fluid, and said measured values are used to determine salinity concentrations of the multiphase fluid.

9. A sensor system for measuring real-time data of a multiphase fluid, comprising:
a vessel sensor, the vessel sensor comprising an inner electrode, an outer cylindrical body, and a cavity located between the inner electrode and the outer cylindrical body, wherein the multiphase fluid flows through the cavity located between the inner electrode and the outer cylindrical body;
an inductor; and
an impedance analyzer.

10. (canceled)

11. The sensor system as recited in claim 9, wherein the vessel sensor provides a response to the multiphase fluid concentration, wherein the multiphase fluid is a down-hole or in-line multiphase fluid in proximity to the vessel sensor.

12. The sensor system as recited in claim 9, wherein the vessel sensor is configured to measure values of real and imaginary parts of an impedance spectra associated with a multiphase fluid concentration while proximate to a multiphase fluid.

13. The sensor system as recited in claim 12, wherein the measured values of real and imaginary parts of the impedance spectra are used to determine salinity concentrations of the multiphase fluid.

14. The sensor system as recited in claim 9, wherein the multiphase fluid is oil, crude oil, desalted oil, or live oil.

15. The sensor system as recited in claim 9, wherein the inductor is a high-Q resonating inductor.

16. The sensor system as recited in claim 9, further comprising a processor coupled to the impedance analyzer.

17. The sensor system as recited in claim 9, wherein the vessel sensor detects a salinity concentration of at least 0.16 lbs/1000 bbl within a multiphase fluid.

18. A method for detecting real-time data of a multiphase fluid, the method comprising:
providing a vessel sensor, wherein the vessel sensor comprises an outer cylindrical body, an inner electrode having an electrode, and a cavity located therebetween;
deploying the vessel sensor within a multiphase fluid environment;
measuring values of both real and imaginary parts of a complex impedance spectra associated with a multiphase fluid; and
determining real-time data of the multiphase fluid.

19. The method as recited in claim 18, wherein the multiphase fluid is a down-hole or in-line multiphase fluid in proximity to the vessel sensor.

20. (canceled)

21. The method as recited in claim 18, wherein the measured values of real and imaginary parts of the impedance spectra associated with the multiphase fluid concentration while proximate to the multiphase fluid are used to determine salinity concentrations of the multiphase fluid.

22. The method as recited in claim 18, wherein the multiphase fluid environment comprises the effluent or influent of a desalter.

23. The method as recited in claim 18, wherein determining the real-time data of the multiphase fluid includes detecting a salinity concentration of at least 0.16 lbs/1000 bbl within the multiphase fluid.
